(19) United States
(12) Patent Application Publication
(10) Pub. No.: US 2025/0026710 A1
(43) Pub. Date: Jan. 23, 2025

(54) PROCESS FOR PREPARING A BENZOATE SALT OF A 1-(3-AMINOPYRROLIDINE-1-CARBONYL)-3,4-DIPHENYLBENZENE COMPOUND

(71) Applicants: Astex Pharmaceuticals, Inc., Pleasanton, CA (US); Taiho Pharmaceutical Co., Ltd., Tokyo (JP)

(72) Inventors: Nipun Davar, Pleasanton, CA (US); Matthew Johnson, Raleigh, NC (US); Suresh Manthati, Sunnyvale, CA (US)

(21) Appl. No.: 18/704,901

(22) PCT Filed: Oct. 25, 2022
(86) PCT No.: PCT/US2022/047766

§ 371 Date: Apr. 25, 2024

Related U.S. Application Data
(60) Provisional application No. 63/271,975, filed on Oct. 26, 2021.

(51) Int. Cl. C07C 67/48; A61K 31/40; A61K 31/4025; C07C 67/52; C07C 69/78; C07D 207/14

(52) U.S. Cl. CPC C07C 67/48; A61K 31/40; A61K 31/4025; C07C 67/52; C07C 69/78; C07D 207/14; C07B 2200/13; C07C 2601/16

(57) ABSTRACT

A process for preparation of benzoate salt of 4-[5-[(3S)-3-aminopyrrolidine-1-carbonyl]-2-[2-fluoro-4-(2-hydroxy-2-methyl-propyl)phenyl]phenyl]-2-fluoro-benzonitrile, designated herein as Compound (A), and methods of using Compound (A) are provided. (I)

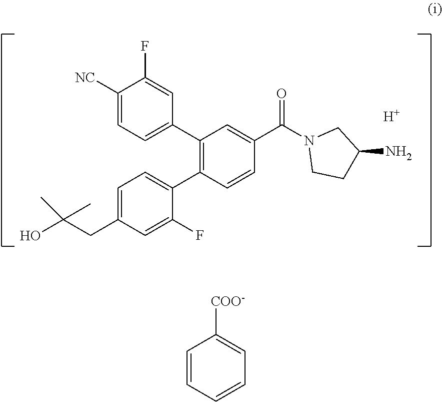

This application claims the benefit under 35 U.S.C. § 119(e) of U.S. Provisional Application No. 63/271,975 filed Oct. 26, 2021, which is hereby incorporated by reference herein in its entirety.

### FIELD

The present disclosure relates generally to processes for making benzoate salt solid forms of compounds that modulate lysine-specific histone demethylase 1A (LSD1), pharmaceutical compositions thereof, and therapeutic uses thereof.

### BACKGROUND

The compound 4-[5-[(3S)-3-aminopyrrolidine-1-carbonyl]-2-[2-fluoro-4-(2-hydroxy-2-methyl-propyl)phenyl]phenyl]-2-fluoro-benzonitrile (referred to herein as Compound (A)) is a potent LSD1 inhibitor and used as an antitumor agent or an agent for preventing and/or treating cancer. The multi-step synthetic routes which have been typically employed for the synthesis of Compound (A), and/or benzoate salt thereof, lead to final products where impurities from various steps are carried into the final product.

It is desired that such an LSD1 inhibitor be prepared reproducibly in high purity and on a large scale.

### SUMMARY

The present disclosure provides a process for preparing a benzoate salt of Compound (A) where a solvent removal step to obtain a dry solid intermediate is avoided by use of a solvent exchange. The process reduces impurities in the final product and is amenable to scale up.

Provided herein is a process for preparing benzoate salt of Compound (A):

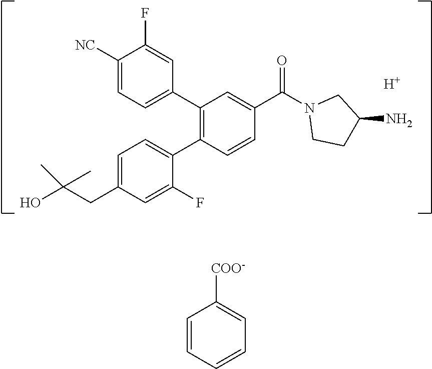

comprising
(i) extracting Compound (A)

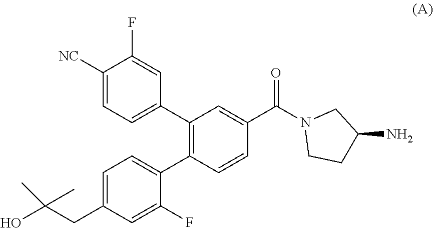 [SMILES: CC(C)(O)Cc1ccc(-c2ccc(C(=O)N3CC[C@H](N)C3)cc2-c2ccc(C#N)c(F)c2)c(F)c1]

into 2-methyl tetrahydrofuran (2-Me THF) from a reaction mixture comprising Compound (A) to obtain a solution of Compound (A) in 2-Me THF;
(ii) conducting a solvent exchange from 2-Me THF to isopropyl alcohol (IPA) to obtain a solution of Compound (A) in IPA; and
(iii) adding benzoic acid to the solution of Compound (A) in IPA under conditions sufficient to obtain benzoate salt of Compound (A).

Also provided herein is benzoate salt of Compound (A) comprising less than 0.5% a/a of certain impurities as described in the detailed description.

### BRIEF DESCRIPTION OF THE DRAWINGS

Embodiments of the present application are described, by way of example only, with reference to the attached Figures, wherein:

FIG. 1A shows an optical microscope image (polarized light microscopy, PLM) of benzoate salt of Compound (A) prepared by the process described herein.

FIG. 1B shows a scanning electron microscope (SEM) image of benzoate salt of Compound (A) prepared by the process described herein.

### DETAILED DESCRIPTION

The compound 4-[5-[(3S)-3-aminopyrrolidine-1-carbonyl]-2-[2-fluoro-4-(2-hydroxy-2-methyl-propyl)phenyl]phenyl]-2-fluoro-benzonitrile, designated herein as Compound (A), has the following formula:

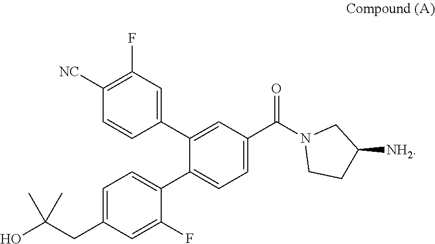 [SMILES: CC(C)(O)Cc1ccc(-c2ccc(C(=O)N3CC[C@H](N)C3)cc2-c2ccc(C#N)c(F)c2)c(F)c1]

Compound (A) is an inhibitor of LSD1. The synthesis and method of use thereof is described in PCT International Application Publication No. WO 2017/090756 which is incorporated by reference herein in its entirety. WO 2021/095835 describes a benzoate salt of Compound (A), and is incorporated by reference herein in its entirety.

In the previously described processes, Compound (A) was initially isolated as a dry solid before conversion to benzoate salt. By contrast, the process described herein involves a solvent exchange that avoids isolation of Compound (A) free base as a solid. The process described herein leads to an overall yield of 20%, is amenable to scale up and is reproducible.

The previously disclosed processes for synthesis of Compound (A) typically result in several impurities telescoped to the final product including, for example, the following impurities:

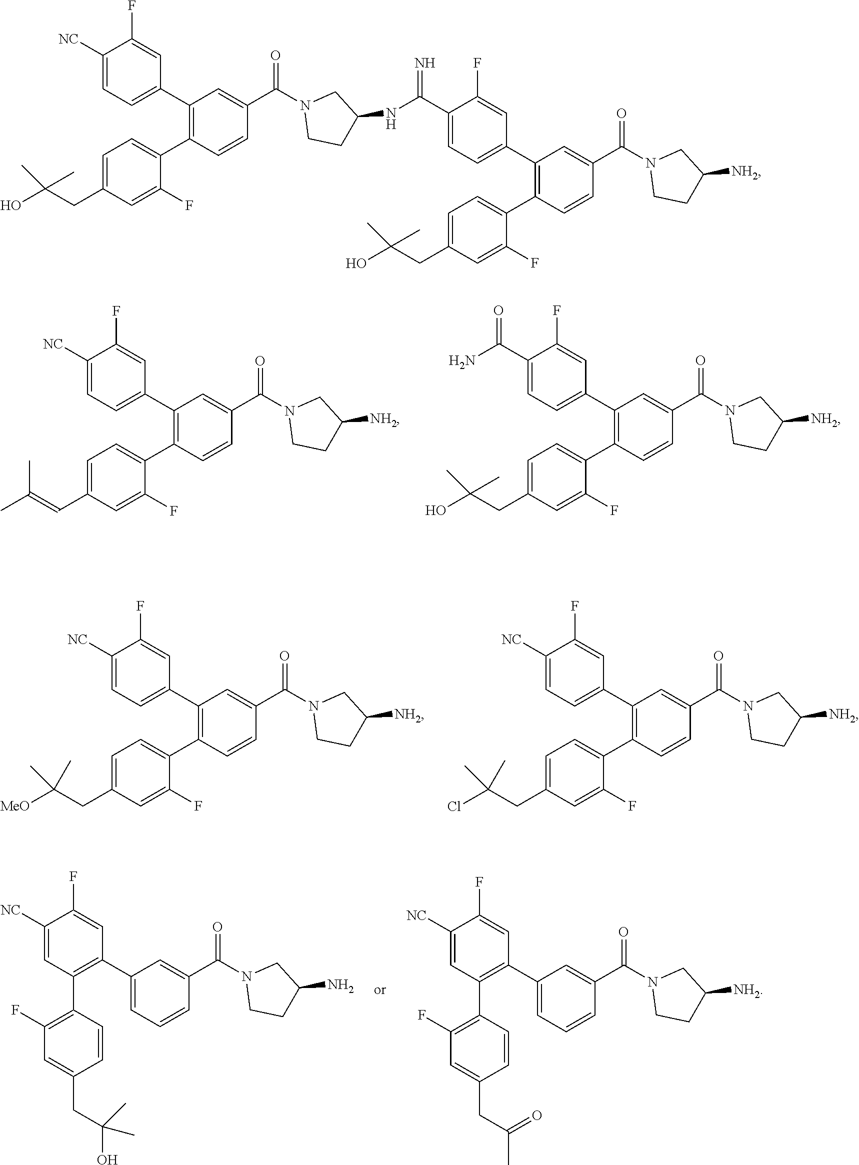

Further, the benzoate salt of Compound (A) obtained by previous processes did not provide optimum morphology and/or desired levels of purity. By contrast, the process described herein reduces the occurrence of impurities and also allows for better control of the morphology of the final benzoate salt product of Compound (A).

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Benzoate salts of Compound (A), and solvates, or hydrates thereof are provided herein. In one embodiment, reference to a form of benzoate salt of Compound (A), and solvate, or hydrate thereof means that at least 95% to 99% (e.g., at least 95%, at least 96%, at least 97%, at least 98%, at least 99%, at least 99.5%, or at least 99.8%) of Compound (A) benzoate salt, solvate, or hydrate thereof present in a composition is in the designated form.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

Any formula or structure given herein, including Compound (A), is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³, ³⁴, ³⁵, ³⁶, ³⁷, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

As used herein, the terms “treat,” “treating,” “therapy,” “therapies,” and like terms refer to the administration of material, e.g., any one or more solid, crystalline or polymorphs of Compound (A) as described herein in an amount effective to prevent, alleviate, or ameliorate one or more symptoms of a disease or condition, i.e., indication, and/or to prolong the survival of the subject being treated.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “modulating” or “modulate” refers to an effect of altering a biological activity, especially a biological activity associated with a particular biomolecule such as LSD1. For example, an agonist or antagonist of a particular biomolecule modulates the activity of that biomolecule, e.g., LSDJ, by either increasing (e.g. agonist, activator), or decreasing (e.g. antagonist, inhibitor) the activity of the biomolecule. Such activity is typically indicated in terms of an inhibitory concentration (IC₅₀) or excitation concentration (EC₅₀) of the compound for an inhibitor or activator, respectively, with respect to, for example, LSD1.

As used herein, the term “LSD1 mediated disease or condition,” refers to a disease or condition in which the biological function of LSD1, including any mutations thereof, affects the development, course, and/or symptoms of the disease or condition, and/or in which modulation of LSD1 alters the development, course, and/or symptoms of the disease or condition. The LSD1 mediated disease or condition includes a disease or condition for which LSD1 modulation provides a therapeutic benefit, e.g. wherein treatment with compound(s), including one or more solid, crystalline or polymorphs of Compound (A) as described herein, provides a therapeutic benefit to the subject suffering from or at risk of the disease or condition.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” or “patient” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

“Substantially pure,” in some embodiments, means that in the referenced material (e.g., Compound (A), or benzoate salt of Compound (A)), at least 99.9% of the material is the referenced polymorph. “Substantially pure,” in some embodiments, means that in the referenced material (e.g., Compound (A), or benzoate salt of Compound (A)), at least 99.5% of the material is the referenced polymorph. “Substantially pure,” in some embodiments, means that in the referenced material (e.g., Compound (A), or benzoate salt of Compound (A)), at least 99% of the material is the referenced polymorph. “Substantially pure,” in some embodiments, means that in the referenced material (e.g., Compound (A), or benzoate salt of Compound (A)), at least 98% of the material is the referenced polymorph. “Substantially pure,” in some embodiments, means that in the referenced material (e.g., Compound (A), or benzoate salt of Compound (A)), at least 97% of the material is the referenced polymorph. “Substantially pure,” in some embodiments, means that in the referenced material (e.g., Compound (A), or benzoate salt of Compound (A)), at least 96% of the material is the referenced polymorph. “Substantially pure,” in some embodiments, means that in the referenced material (e.g., Compound (A), or benzoate salt of Compound (A)), at least 95% of the material is the referenced polymorph.

“Substantially free of aqueous NaOH” means that the mixture of (A) in 2-Me THF (e.g., a mixture comprising (A) dissolved in 2-Me THF) after step (i-g) as described herein has less than 5%, less than 1% or less than 0.1% by weight of NaOH in the mixture compared to the total weight of the mixture.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

In addition, abbreviations as used herein have respective meanings as follows:
ACN or MeCN Acetonitrile
Aq Aqueous
(Boc)₂O Di-tert-butyl dicarbonate
B₂(Pin)₂ Bis(pinacolato)diboron
DMA Dimethylacetamide
DMAP Dimethylamino pyridine
DME Dimethylether
Et₃N Triethylamine
EtOAc Ethyl acetate
EtOH Ethanol
GC Gas Chromatography
HATU 1-[Bis(dimethylamino)methylene]-1H-1,2,3-triazolo [4,5-b]pyridinium 3-oxid hexafluorophosphate
HPLC High Pressure Liquid Chromatography
IPA Isopropyl alcohol or 2-propanol
IPAc Isopropyl acetate
LC/MS Liquid chromatography mass spectrometry
2-Me THF 2-methyl tetrahydrofuran
MeOH Methanol
MEK Methyl ethyl ketone
MTBE Methyl tert-butyl ether
NMR Nuclear magnetic resonance spectroscopy
nPrOH n-Propanol
P parts by weight or weight equivalent(s)
PdCl₂(dppf) [1,1′-Bis (diphenylphosphino)ferrocene]dichloropalladium(II)
Pd₂(dba)₂ Bis(dibenzylideneacetone)palladium(0)
Pd₂dba₃ Tris(dibenzylideneacetone)dipalladium(0)
PhCO₂H Benzoic acid
RT Room temperature
RRT Relative retention time
S-Phos 2-Dicyclohexylphosphino-2′,6′-dimethoxybiphenyl,
TMSOTf Trimethylsilyl triflate
THF Tetrahydrofuran
v Volume(s)
v/v volume to volume
w/w weight to weight
X-Phos 2-Dicyclohexylphosphino-2′,4′,6′-triisopropylbiphenyl

### 2. Methods of Preparing Benzoate Salt of Compound (A)

In some embodiments, provided herein is a process for preparing benzoate salt of Compound (A):

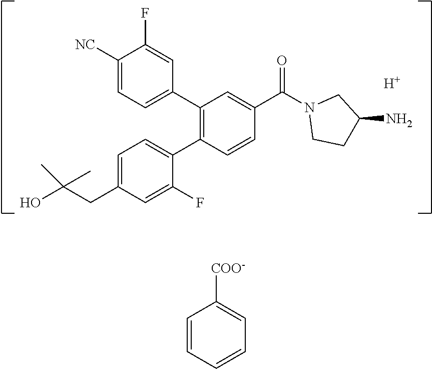

comprising
(i) extracting Compound (A) into 2-methyl tetrahydrofuran (2-Me THF) from a reaction mixture comprising Compound (A) to obtain a solution of Compound (A) in 2-Me THF; and
(ii) conducting a solvent exchange from 2-Me THF to isopropyl alcohol (IPA) to obtain a solution of Compound (A) in IPA; wherein the peroxide content of the IPA is no more than 30 ppm, no more than 20 ppm, or no more than 10 ppm.

In one embodiment, provided herein is a process for preparing benzoate salt of Compound (A):

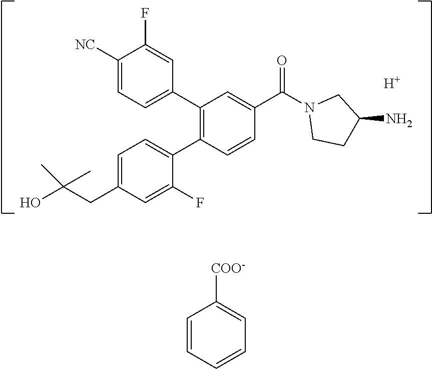

comprising
(i) extracting Compound (A)

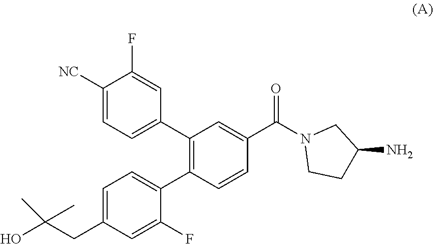 [SMILES: CC(C)(O)Cc1ccc(-c2ccc(C(=O)N3CC[C@H](N)C3)cc2-c2ccc(C#N)c(F)c2)c(F)c1]

into 2-methyl tetrahydrofuran (2-Me THF) from a reaction mixture comprising Compound (A) to obtain a solution of Compound (A) in 2-Me THF;
(ii) conducting a solvent exchange from 2-Me THF to isopropyl alcohol (IPA) to obtain a solution of Compound (A) in IPA; and
(iii) adding benzoic acid to the solution of Compound (A) in IPA under conditions sufficient to obtain Compound (A) benzoate salt.

In one embodiment, the process further comprises recrystallizing the benzoate salt of Compound (A). The crystallization may be carried out in IPA, or any other suitable solvent. Optionally, a second recrystallization may be carried out.

In some embodiments, the peroxide content of the IPA is no more than 10 ppm prior to introduction of the IPA in the reaction mixture. In some embodiments, the peroxide content of the IPA is no more than 20 ppm prior to introduction of the IPA in the reaction mixture. In some embodiments, the peroxide content of the IPA is no more than 30 ppm prior to introduction of the IPA in the reaction mixture.

In some embodiments, the Compound (A) in a reaction mixture is obtained by contacting Compound (B)

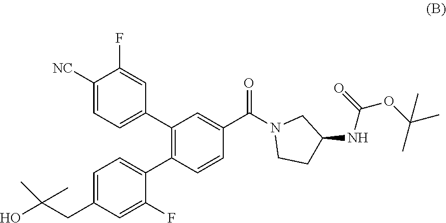 [SMILES: CC(C)(O)Cc1ccc(-c2ccc(C(=O)N3CC[C@H](NC(=O)OC(C)(C)C)C3)cc2-c2ccc(C#N)c(F)c2)c(F)c1]

with hydrochloric acid in a solvent thereby providing Compound (A) in the reaction mixture. In some embodiments, the solvent is methanol. In some embodiments, the solvent is water, methanol, ethanol, isopropyl alcohol, or tetrahydrofuran.

In some embodiments, the reaction mixture wherein Compound (B) is deprotected to provide Compound (A) is heated at a temperature of from about 15° C. to about 50° C. In some embodiments, the reaction mixture wherein Compound (B) is deprotected to provide Compound (A) is heated at a temperature of from about 40° C. to about 50° C. In some of these embodiments, the reaction mixture wherein Compound (B) is deprotected to provide Compound (A) is stirred for a period of about 1 hour to about 8 hours. In some of these embodiments, the reaction mixture wherein Compound (B) is deprotected to provide Compound (A) is stirred for a period of about 3 hours to about 6 hours. In some of these embodiments, the reaction mixture wherein Compound (B) is deprotected to provide Compound (A) is stirred for a period of about 5 hours.

In some embodiments, the process wherein Compound (B) is deprotected to provide Compound (A) comprises:
(i-a) cooling the reaction mixture described above and adding water and methyl tert butyl ether (MTBE) to the reaction mixture;
(i-b) separating the organic layer comprising MTBE and the aqueous layer;
(i-c) adding 2-Me THF to the aqueous layer from step (i-b) and adding aqueous NaOH to the mixture comprising 2-Me THF and the aqueous layer;
(i-d) separating the organic layer comprising 2-Me THF; and
(i-e) washing the organic layer of step (i-d) with 20% w/w NaCl solution;
(i-f) washing the organic layer of step (i-e) with water; and
(i-g) optionally repeating step (i-f) once, or more than once, to obtain a solution of Compound (A) in 2 Me-THF.

In some embodiments, in step (i-a), the reaction mixture is cooled to a temperature of about 20° C. to about 25° C. and is optionally stirred for a period of about 1 hour to about 24 hours, about 1 hour to about 12 hours, about 1 hour to about 6 hours, or about 1 hour to about 4 hours prior to adding water and MTBE. In some embodiments, in step (i-a), the reaction mixture is cooled to a temperature of about 20° C. to about 25° C. and is optionally stirred for a period of about 2 hours to about 24 hours prior to adding water and MTBE. In some embodiments, in step (i-a), the reaction mixture is cooled to a temperature of about 20° C. to about 25° C. and is optionally stirred for a period of about 1 hour to about 2 hours prior to adding water and MTBE. In some embodiments, in step (i-a), the reaction mixture is cooled to a temperature of about 20° C. to about 25° C. and is optionally stirred for a further period till the reaction is complete prior to adding water and MTBE.

In some embodiments, in step (i-c), the aqueous NaOH is added to the mixture to a pH of at least pH 13. In some embodiments, the pH is at least 12. In some embodiments, the pH is at least 11. In some embodiments, the pH is at least 10. In some embodiments, the pH is at least 9. After separating the layers as described in step (i-d), the organic layer is washed with an NaCl solution, the layers are separated, and the organic layer is then washed with water. This washing of the organic layer with water as described in step (i-f) removes NaOH from the organic layer that is obtained after step (i-e). One or more than one wash with water may be required to obtain an organic layer, comprising a solution of Compound (A) in 2-Me THF, that is substantially free of NaOH. In some embodiments, after step (i-g), the solution of Compound (A) in 2-Me THF is substantially free of aqueous NaOH. In some embodiments, in step (i-c), the aqueous NaOH is added to the mixture to a basic pH in a range of about 7 to about 13, about 8 to about 13, about 9 to about 13, about 10 to about 13, about 11 to about 13, or about 12 to about 13. In some embodiments, the pH of the mixture at the time of addition of aqueous NaOH affects the overall yield of the process for making Compound (A) benzoate salt. In some embodiments, removal of NaOH so that any residual NaOH is less than 0.1% w/w of the mixture affects the overall yield of the process for making Compound (A) benzoate salt.

In some embodiments, in step (iii), where Compound (A) is converted to benzoate salt of Compound (A), step (iii) further comprises heating the reaction mixture to a temperature of about 70° C. to about 80° C. and maintaining the reaction mixture at about 70° C. to about 80° C. for a period of about 30 minutes to about 60 minutes. In some embodiments, the heated reaction mixture is cooled to a temperature of about 50° C. to about 55° C. In some embodiments, a slurry of seed crystal of benzoate salt of Compound (A) in IPA is added to the cooled reaction mixture. In some embodiments, the seed crystals are prepared according to methods described in PCT publication WO 2021/095835.

In some embodiments, after adding seed crystals, the reaction mixture is subjected to cooling and heating cycles, then cooled and stirred at the cooled temperature for about 4 to 10 hours (e.g., 6 hours). In some embodiments, the cooling and heating cycles comprise an initial cooling cycle where
(iii-a) the reaction mixture is cooled to a temperature of about 10° C. to about 30° C., about 16° C. to about 24° C., or about 20° C., over a period of about 150 to about 400 minutes, about 200 to about 300 minutes, or about 200 minutes to about 250 minutes, and then stirred at the cooled temperature for about 20 minutes, about 30 minutes, about 45 minutes or about 60 minutes.

In some embodiments, the cooling and heating cycles comprise a cycle where
(iii-b) the reaction mixture is heated to a temperature of about 60° C. to about 75° C., about 65° C. to about 70° C. or about 62° C. to about 66° C. over a period of about 200 minutes to about 400 minutes, about 250 minutes to about 350 minutes, or about 280 minutes to about 320 minutes.

In some embodiments, the cooling and heating cycles comprise additional cycles where
(iii-c) the cooling and heating of steps (iii-a) and (iii-b) are repeated one or two time(s).

In some embodiments, the cooling and heating cycles comprise additional cycles where
(iii-d) the reaction mixture is cooled to a temperature of about 10° C. to about 30° C., about 16° C. to about 24° C., or about 20° C., over a period of about 150 to about 400 minutes, about 250 to about 350 minutes, or about 280 minutes to 320 minutes, and then stirred at the cooled temperature of about 10° C. to about 30° C., about 16° C. to about 24° C., or about 20° C., for about 2 to about 10 hours, about 4 to about 8 hours, or about 6 hours.

In some embodiments, after adding seed crystals, the reaction mixture is subjected to cooling and heating cycles, then cooled to a temperature of about 16° C. to about 24° C. and stirred at the temperature of about 16° C. to about 24° C. for about 6 hours. In some embodiments, the cooling and heating cycles comprise steps where
(iii-a) the reaction mixture is cooled to a temperature of about 16° C. to about 24° C. over a period of about 200 minutes to about 250 minutes and then stirred at the temperature of about 16° C. to about 24° C. for about 30 minutes;
(iii-b) the reaction mixture is heated to a temperature of about 62° C. to about 66° C. over a period of about 280 minutes to about 320 minutes;
(iii-c) the cooling and heating of steps (iii-a) and (iii-b) is repeated one or two times; and
(iii-d) the reaction mixture is cooled to a temperature of about 16° C. to about 24° C. over a period of about 280 minutes to 320 minutes and stirred at the temperature of about 16° C. to about 24° C. for about 6 hours.

In some embodiments of the process described herein, the benzoate salt of Compound (A) is recrystallized from ethanol, butanol, IPA, acetonitrile, TBME, ethyl acetate, isopropyl acetate, methylethyl ketone (MEK), proprionitrile, or toluene. In some embodiments of the process described herein, benzoate salt of Compound (A) is initially crystallized from IPA, then subjected to a further recrystallization from IPA, or any other solvent including and not limited to ethanol, butanol, acetonitrile, TBME, ethyl acetate, isopropyl acetate, methylethyl ketone (MEK), proprionitrile, or toluene.

In some embodiments of the process described herein, the recrystallized benzoate salt of Compound (A) has no more than 0.7% a/a, or no more than 0.5% a/a, or no more than 0.3% a/a, total, of any of the following compounds, or a combination thereof:

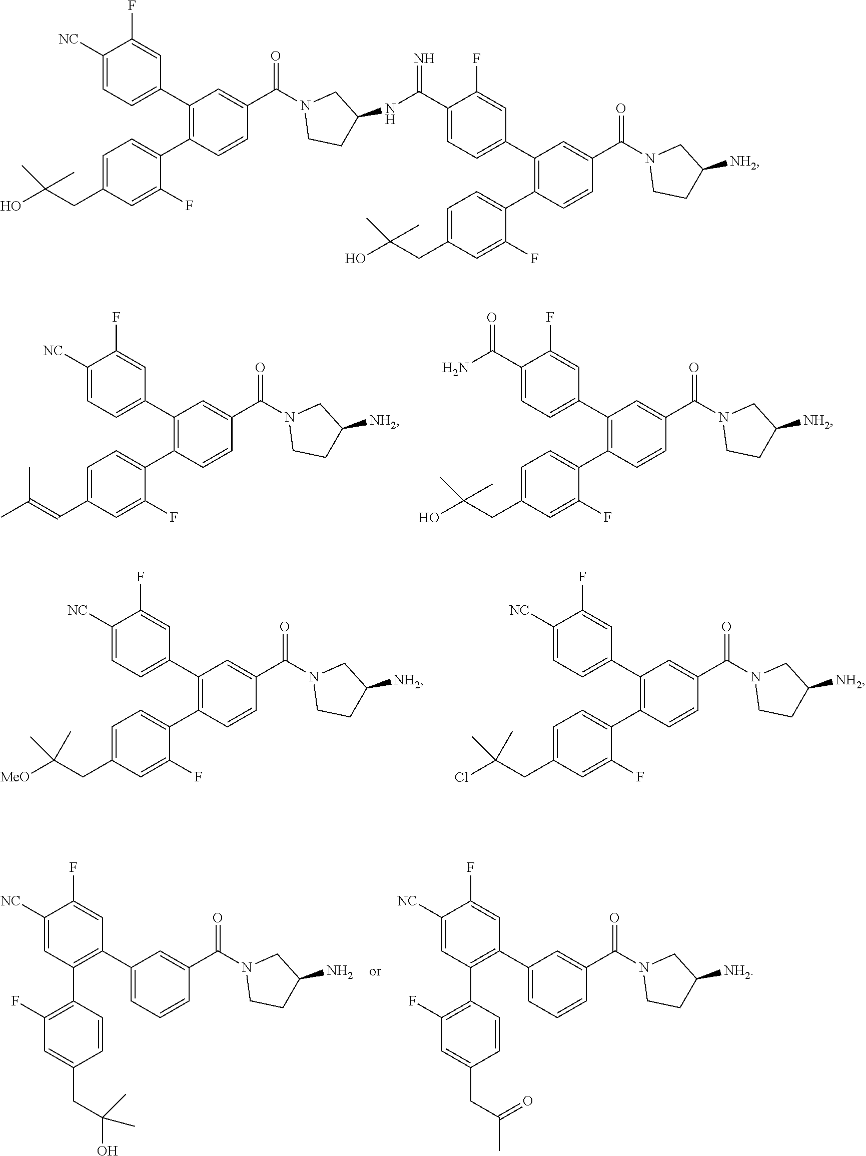

In some embodiments provided is a process for preparing benzoate salt of Compound (A):

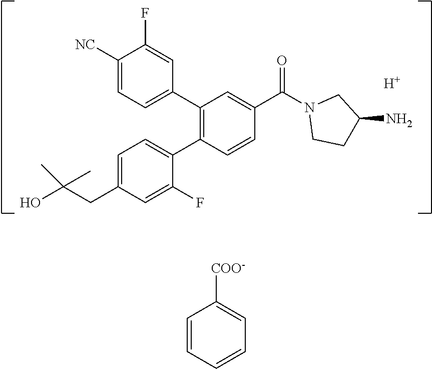

comprising
(i) contacting Compound (B)

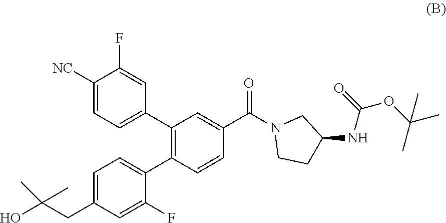 [SMILES: CC(C)(O)Cc1ccc(-c2ccc(C(=O)N3CC[C@H](NC(=O)OC(C)(C)C)C3)cc2-c2ccc(C#N)c(F)c2)c(F)c1]

with aqueous hydrochloric acid to obtain Compound (A)

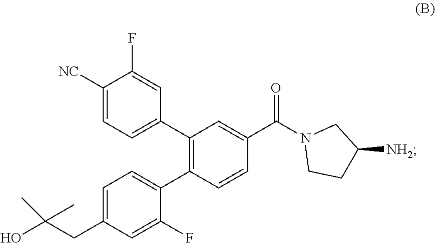 [SMILES: CC(C)(O)Cc1ccc(-c2ccc(C(=O)N3CC[C@H](N)C3)cc2-c2ccc(C#N)c(F)c2)c(F)c1]

(ii) extracting Compound (A) into 2-methyl tetrahydrofuran (2-Me THF) to obtain a solution of Compound (A) in 2-Me THF;
(iii) conducting a solvent exchange from 2-Me THF to isopropyl alcohol (IPA);
(iv) adding benzoic acid to the mixture comprising IPA under conditions sufficient to obtain benzoate salt of Compound (A); and
(v) recrystallizing the benzoate salt of Compound (A).

Provided herein is benzoate salt of Compound (A) prepared according to any process described herein.

Provided herein is benzoate salt of Compound (A) comprising no more than 0.7% a/a, or no more than 0.5% a/a, or no more than 0.3% a/a, total, of any of the following compounds or a combination thereof:

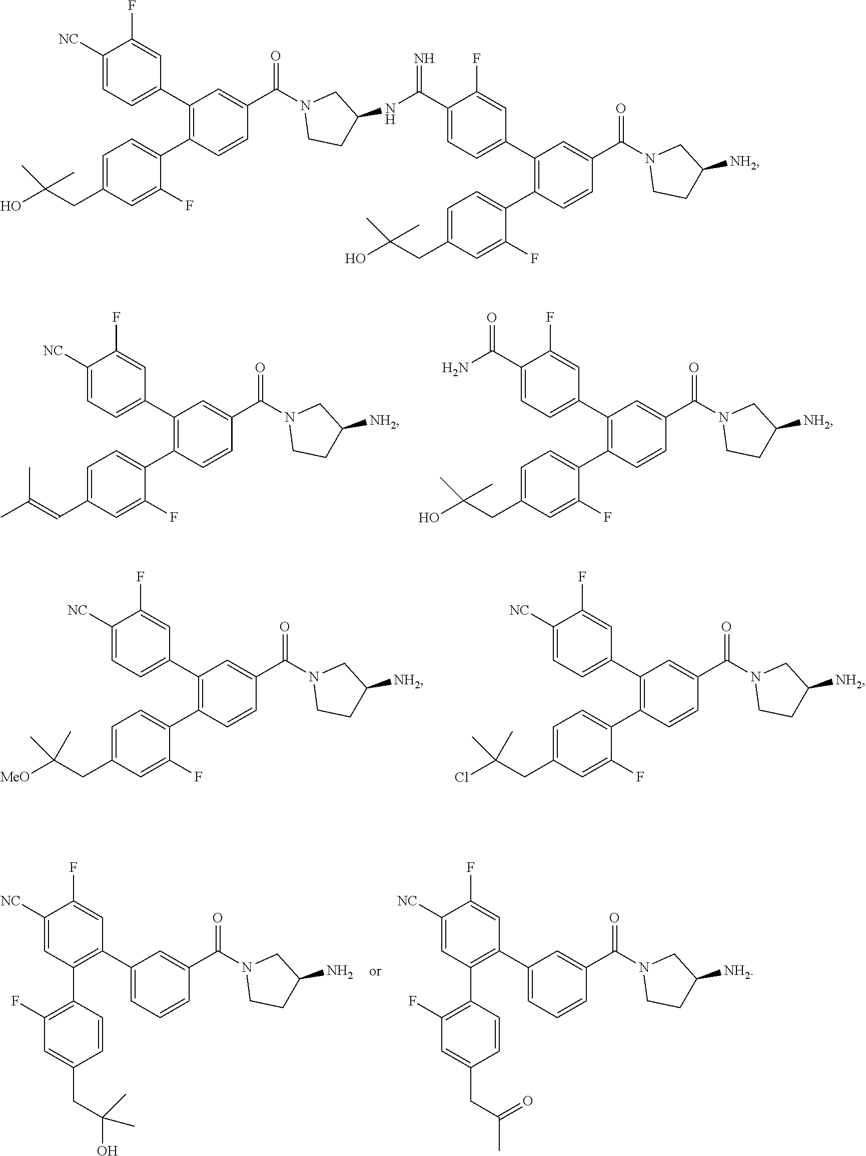

In some embodiments, provided herein is a process for preparing Compound (A) comprising:
contacting Compound (C), or a salt thereof,

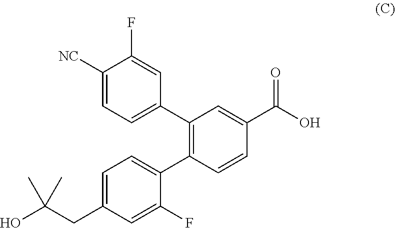 [SMILES: CC(C)(O)Cc1ccc(-c2ccc(C(=O)O)cc2-c2ccc(C#N)c(F)c2)c(F)c1]

with Compound (D), or a salt thereof,

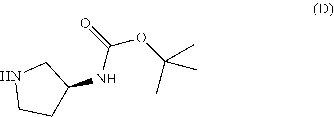

under conditions sufficient to provide Compound (B), or a salt thereof,

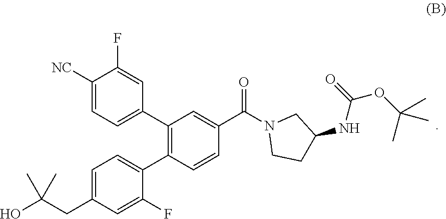 [SMILES: CC(C)(O)Cc1ccc(-c2ccc(C(=O)N3CC[C@H](NC(=O)OC(C)(C)C)C3)cc2-c2ccc(C#N)c(F)c2)c(F)c1]

In some embodiments, provided herein is a process for preparing Compound (C), or a salt thereof,

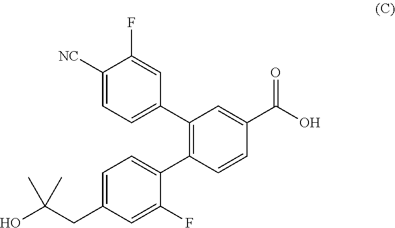 [SMILES: CC(C)(O)Cc1ccc(-c2ccc(C(=O)O)cc2-c2ccc(C#N)c(F)c2)c(F)c1]

comprising:
(i) contacting Compound (G), or a salt thereof,

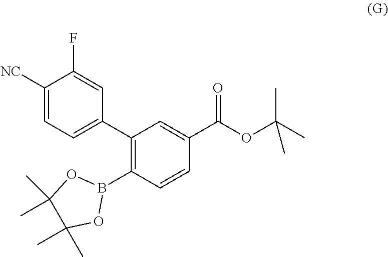 [SMILES: CC(C)(C)OC(=O)c1ccc(B2OC(C)(C)C(C)(C)O2)c(-c2ccc(C#N)c(F)c2)c1]

with Compound (F), or a salt thereof,

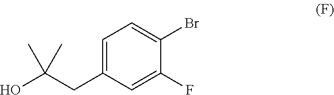

under conditions sufficient to provide Compound (E), or a salt thereof,

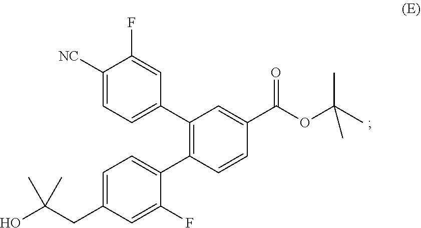 [SMILES: CC(C)(O)Cc1ccc(-c2ccc(C(=O)OC(C)(C)C)cc2-c2ccc(C#N)c(F)c2)c(F)c1]

and
(ii) deprotecting Compound (E), or a salt thereof, to provide Compound (C), or a salt thereof.

In some embodiments, provided herein is a process for preparing Compound (G), or a salt thereof,

comprising:

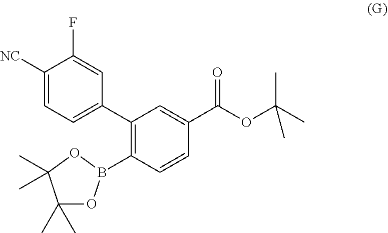 [SMILES: CC(C)(C)OC(=O)c1ccc(B2OC(C)(C)C(C)(C)O2)c(-c2ccc(C#N)c(F)c2)c1]

(i) contacting Compound (L), or a salt thereof,

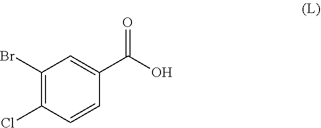 [SMILES: O=C(O)c1ccc(Cl)c(Br)c1]

with Compound (K), or a salt thereof,

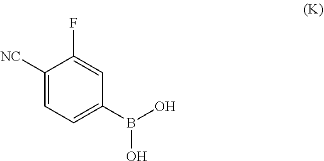 [SMILES: N#Cc1ccc(B(O)O)cc1F]

under conditions sufficient to provide Compound (J), or a salt thereof,

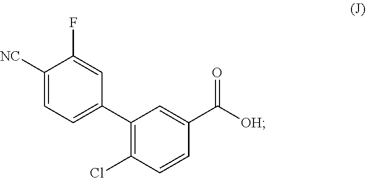 [SMILES: N#Cc1ccc(-c2cc(C(=O)O)ccc2Cl)cc1F]

(ii) protecting Compound (J), or a salt thereof, to provide Compound (H), or a salt thereof,

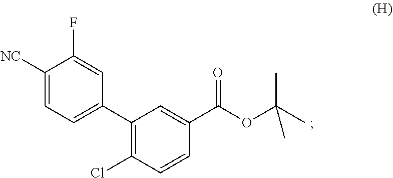 [SMILES: CC(C)(C)OC(=O)c1ccc(Cl)c(-c2ccc(C#N)c(F)c2)c1]

and
(iii) borylating Compound (H), or a salt thereof, to provide Compound (G), or a salt thereof.

In some embodiments, provided herein is a method for preparing Compound (A) according to Scheme 1. Compound (A) can be converted to benzoate salt of Compound (A) according to the methods described herein.

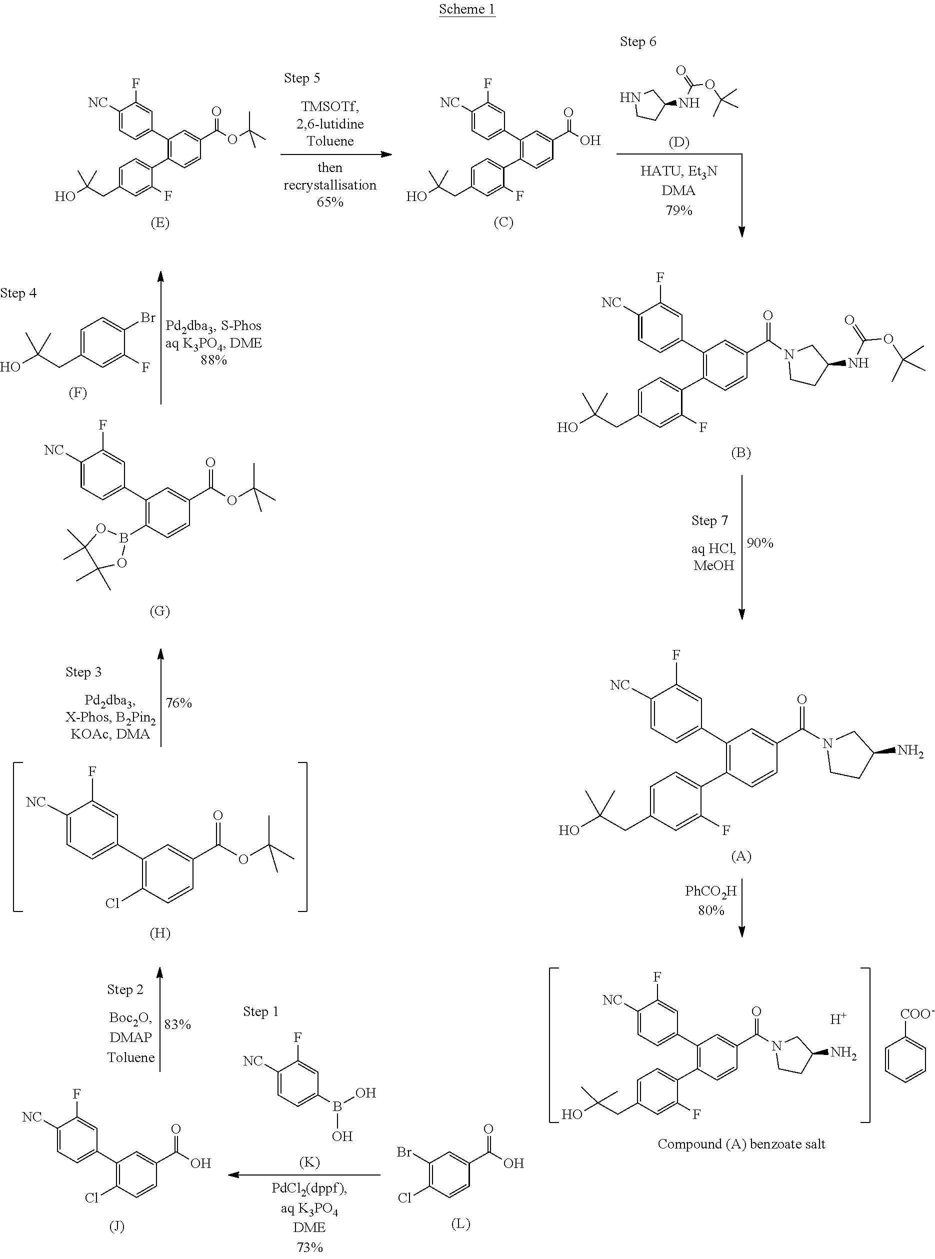

Referring to Scheme 1 above, Compound (C) and other intermediates can be prepared according to methods described in U.S. Pat. No. 10,723,742, and such methods are incorporated herein by reference. Compound (C) is coupled with Compound (D) to provide Compound (B). Compound (B) is deprotected to provide Compound (A), and Compound (A) is converted to benzoate salt of Compound (A) using the methods described herein.

### 3. Pharmaceutical Compositions, Kits, and Modes of Administration

Compound (A) or benzoate salt thereof as described herein may be administered in a pharmaceutical composition. In some embodiments, provided herein is a pharmaceutical composition comprising benzoate salt of Compound (A) prepared according to any process described herein.

In some embodiments, provided herein is a pharmaceutical composition comprising benzoate salt of Compound (A) and comprising no more than 0.7% a/a, or no more than 0.5% a/a, or no more than 0.3% a/a, total, of any of the following compounds or a combination thereof:

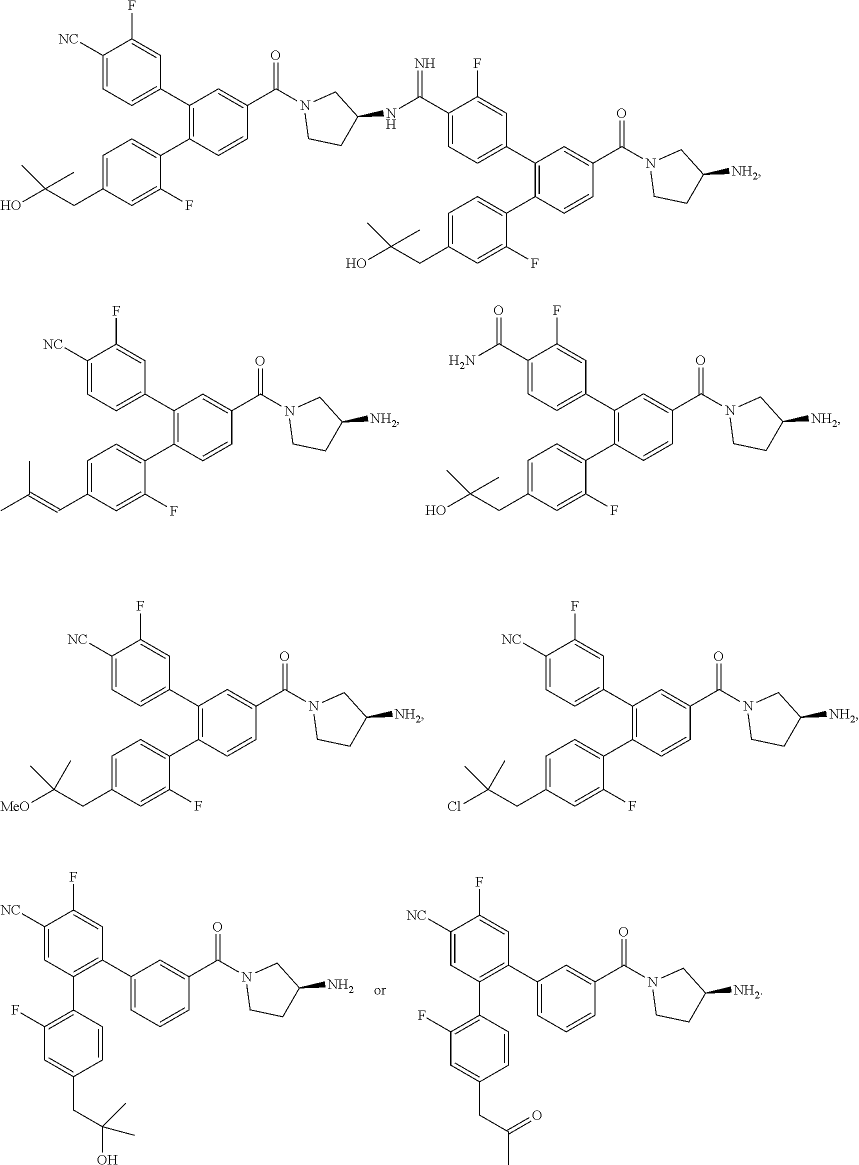

In some embodiments, provided herein is a pharmaceutical composition comprising benzoate salt of Compound (A) and comprising no more than 4.0% a/a of total impurities.

In some embodiments, provided herein are pharmaceutical compositions comprising Compound (A) or benzoate salt thereof described herein and one or more pharmaceutically acceptable vehicles such as carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.). The pharmaceutical compositions may be administered alone or in combination with other therapeutic agents.

Some embodiments are directed to pharmaceutical compositions comprising a therapeutically effective amount of Compound (A) or benzoate salt thereof described herein.

Some embodiments are directed to pharmaceutical compositions comprising Compound (A) or benzoate salt thereof as described herein and one or more pharmaceutically acceptable carriers. In one embodiment, a pharmaceutical composition comprises Compound (A), wherein at least 95% of Compound (A) is benzoate salt of Compound (A) as described herein. In one embodiment, a pharmaceutical composition comprises Compound (A), wherein at least 96% of Compound (A) is benzoate salt of Compound (A) as described herein. In one embodiment, a pharmaceutical composition comprises Compound (A), wherein at least 97% of Compound (A) is benzoate salt of Compound (A) as described herein. In one embodiment, a pharmaceutical composition comprises Compound (A), wherein at least 98% of Compound (A) is benzoate salt of Compound (A) as described herein. In one embodiment, a pharmaceutical composition comprises Compound (A), wherein at least 99% of Compound (A) is benzoate salt of Compound (A) as described herein. In one embodiment, a pharmaceutical composition comprises Compound (A), wherein at least 99.5% of Compound (A) is benzoate salt of Compound (A) as described herein. In one embodiment, a pharmaceutical composition comprises Compound (A), wherein at least 99.9% of Compound (A) is benzoate salt of Compound (A) as described herein.

Benzoate salt of Compound (A) described herein may be processed, after being pulverized or without being pulverized, into various forms of pharmaceutical composition, for example tablets, capsules, granules, fine granules, powdered drug, dry syrup and like oral preparations, suppositories, inhalation agents, nasal drops, ointments, patches, aerosols, and the like.

In some embodiments, compositions comprise pharmaceutically acceptable carriers or excipients, such as fillers, binders, disintegrants, glidants, lubricants, complexing agents, solubilizers, and surfactants, which may be chosen to facilitate administration of the Compound (A) by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopherol polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the present disclosure (as a free-acid, solvate (including hydrate) or salt, in any form), depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical formulations may be prepared by any of the methods well known in the pharmacy art.

Compound (A) or benzoate salt thereof is usually administered in the form of pharmaceutical compositions. Thus, provided herein are also pharmaceutical compositions that contain Compound (A) or benzoate salt thereof as described herein and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

When an injection is prepared, a pH adjuster, a buffer, a stabilizer, an isotonizing agent, a topical anesthetic, and the like may be added, as necessary, to the crystalline form of Compound (A); and the resulting mixture may be formulated into subcutaneous, intramuscular, and intravenous injections according to an ordinary method.

Examples of usable pH adjusters and buffers include sodium citrate, sodium acetate, sodium phosphate, and the like. Examples of usable stabilizers include sodium pyrosulfite, EDTA, thioglycolic acid, and thiolactic acid. Examples of usable topical anesthetics include procaine hydrochloride, lidocaine hydrochloride, and the like. Examples of usable isotonizing agents include sodium chloride, glucose, D-mannitol, glycerin, and the like.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, sterile injectable solutions, and sterile packaged powders.

Oral solid preparations may be prepared as follows. After an excipient is added optionally with a binder, disintegrant, lubricant, colorant, taste-masking or flavoring agent, etc., to a crystalline form of Compound (A), the resulting mixture is formulated into tablets, coated tablets, granules, powders, capsules, or the like by methods known in the art.

Examples of excipients include lactose, sucrose, D-mannitol, glucose, starch, calcium carbonate, kaolin, microcrystalline cellulose, and silicic acid anhydride. Examples of binders include water, ethanol, 1-propanol, 2-propanol, simple syrup, liquid glucose, liquid α-starch, liquid gelatin, D-mannitol, carboxymethyl cellulose, hydroxypropyl cellulose, hydroxypropyl starch, methyl cellulose, ethyl cellulose, shellac, calcium phosphate, polyvinylpyrrolidone, and the like. Examples of disintegrators include dry starch, sodium alginate, powdered agar, sodium hydrogen carbonate, calcium carbonate, sodium lauryl sulfate, stearic acid monoglyceride, lactose, and the like. Examples of lubricants include purified talc, stearic acid salt sodium, magnesium stearate, borax, polyethylene glycol, and the like. Examples of colorants include titanium oxide, iron oxide, and the like. Examples of taste-masking or flavoring agents include sucrose, bitter orange peel, citric acid, L-tartaric acid, and the like. The formulations can additionally include wetting agents; emulsifying and suspending agents; preserving agents such as methyl and propylhydroxy-benzoates.

When a liquid preparation for oral administration is prepared, a taste-masking agent, a buffer, a stabilizer, a flavoring agent, and the like may be added to Compound (A) or benzoate salt thereof described herein and the resulting mixture may be formulated into an oral liquid preparation, syrup, elixir, etc., according to an ordinary method.

In this case, the same taste-masking or flavoring agent as those mentioned above may be used. Examples of the buffer include sodium citrate and the like, and examples of the stabilizer include tragacanth, gum arabic, gelatin, and the like. As necessary, these preparations for oral administration may be coated according to methods known in the art with an enteric coating or other coating for the purpose of, for example, persistence of effects. Examples of such coating agents include hydroxypropyl methylcellulose, ethyl cellulose, hydroxymethyl cellulose, hydroxypropyl cellulose, polyoxyethylene glycol, and Tween 80®.

The compositions that include Compound (A) or benzoate salt thereof as described herein, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of Compound (A) or benzoate salt thereof as described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of Compound (A) or benzoate salt thereof as described herein, may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In another aspect, the present disclosure provides kits or containers that include a Compound (A) or benzoate salt thereof and any of its forms as described herein, or any of the pharmaceutical compositions thereof described herein. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human, for a bromodomain protein mediated disease or condition; the kit or container disclosed herein may include written instructions for use and/or other indication that the compound or composition is suitable or approved for administration to a mammal, e.g., a human, for a bromodomain-mediated disease or condition; and the compound or composition may be packaged in unit dose or single dose form, e.g., single dose pills, capsules, or the like.

The amounts of various compounds to be administered can be determined by standard procedures taking into account factors such as the compound activity (in vitro, e.g. the C₅₀ of Compound (A) vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose will be in the range of about 0.01 to 50 mg/kg, also about 0.1 to 20 mg/kg of the subject being treated. Multiple doses may be used.

The amount of Compound (A) or benzoate salt thereof to be incorporated in each of such dosage unit forms depends on the condition of the patient to whom the Compound (A) or benzoate salt thereof is to be administered, the dosage form, etc. In general, in the case of an oral agent, an injection, and a suppository, the amount of the compound of the present disclosure is preferably 0.05 to 1000 mg, 0.01 to 500 mg, and 1 to 1000 mg, respectively, per dosage unit form.

The daily dose of the medicine in such a dosage form depends on the condition, body weight, age, gender, etc., of the patient, and cannot be generalized. For example, the daily dose of a salt of Compound (A) described herein for an adult (body weight: 50-70 kg kg) may be 0.05 to 5000 mg, or 0.1 to 1000 mg; and may be administered in one dose, or in two to four divided doses, per day, or any other suitable dosing schedule.

### 4. Dosing

The specific dose level of Compound (A) or benzoate salt thereof as described herein, for any particular subject, will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 5. Disease Indications and Modulation of LSD1

Provided herein is a method for treating a lysine-specific histone demethylase 1A (LSD-1) related disease or condition in a mammal, the method comprising administering to the mammal a therapeutically effective amount of Compound (A) or benzoate salt thereof described herein, or a composition described herein.

In some embodiments, the LSD-1 related disease or condition is cancer.

In some embodiments, the cancer is a malignant tumor.

In some embodiments, the cancer is head and neck cancer, esophagus cancer, gastric cancer, colon cancer, rectum cancer, liver cancer, gallbladder cancer, cholangiocarcinoma, biliary tract cancer, pancreatic cancer, lung cancer, breast cancer, ovarian cancer, cervical cancer, endometrial cancer, renal cancer, bladder cancer, prostate cancer, testicular tumor, osteosarcoma, soft-tissue sarcoma, leukemia, myelodysplastic syndrome, chronic myeloproliferative disease, malignant lymphoma, multiple myeloma, skin cancer, brain tumor, or mesothelioma.

In some embodiments, the cancer is non-small cell lung cancer, small cell lung cancer, leukemia, or myelodysplastic syndromes. In some embodiments, the cancer is acute myeloid leukemia (AML), including relapsed or refractory (r/r) AML.

In certain embodiments, the present disclosure provides use of Compound (A) or benzoate salt thereof as described herein, or any of the pharmaceutical compositions thereof described herein in the manufacture of a medicament for the treatment of a disease or condition as described herein. In other embodiments, the present disclosure provides Compound (A) or benzoate salt thereof as described herein, or any of the pharmaceutical compositions thereof described herein for use in treating a disease or condition as described herein.

### EXAMPLES

This disclosure is further understood by reference to the following examples, which are intended to be purely exemplary of this disclosure. This disclosure is not limited in scope by the exemplified embodiments, which are intended as illustrations of single aspects of this disclosure only. Any methods that are functionally equivalent are within the scope of this disclosure. Various modifications of this disclosure in addition to those described herein will become apparent to those skilled in the art from the foregoing description and accompanying figures. Such modifications fall within the scope of the appended claims.

The starting materials may be generally known compounds or can be prepared by known procedures or obvious modifications thereof. For example, many of the starting materials are available from commercial suppliers such as Sigma Aldrich (St. Louis, Missouri, USA), Bachem (Torrance, California, USA), Emka-Chemce (St. Louis, Missouri, USA). Others may be prepared by procedures, or obvious modifications thereof, described in standard reference texts such as Fieser and Fieser's Reagentsfor Organic Synthesis, Volumes 1-15 (John Wiley, and Sons, 2016), Rodd's Chemistry of Carbon Compounds, Volumes 1-5, and Supplementals (Elsevier Science Publishers, 2001), Organic Reactions, Volumes 1-40 (John Wiley, and Sons, 2019), March's Advanced Organic Chemistry, (John Wiley, and Sons, 8^{th} Edition, 2019), and Larock's Comprehensive Organic Transformations (VCH Publishers Inc., 1989).

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

### Example 1: Preparation of Benzoate Salt of Compound (A)

In a jacketed reactor equipped with mechanical stirring, was added Compound (B) and 4.5 v of methanol. The mixture was heated to about 45° C. if necessary to dissolve Compound (B) and then cooled to about 35° C. In an auxiliary vessel was prepared an HCl 6M solution mixing 1.2 v of water and 1.2 v of HCl 35% w/w. 1.3 vol of the prepared HCl 6M was charged to the main vessel while maintaining the temperature of the reaction at about 35° C.

The reaction mixture was heated to about 45° C. (about 40° C. to about 50° C.) and stirred at about 45° C. (about 40° C. to about 50° C.) for about 4-5 hours and tested by HPLC to determine if reaction was complete and Compound (A) was formed. The reaction mixture was cooled to about 15° C. to about 25° C. while running the HPLC. If the in-process-control indicated that >99% of starting material was consumed, the reaction was quenched as follows.

The reaction mixture was maintained at about 20 to 30° C. while charging 10 v of water, carefully maintaining temperature below 30° C. (heat release was observed). The reaction mixture was cooled to about 15° C. to about 25° C.

5 v of MTBE was then charged to the reactor while maintaining the temperature below 30° C. The reaction mixture was cooled to about 15° C. to about 25° C. and stirred for about 15 minutes while maintaining the temperature at about 15° C. to about 25° C. (e.g., about 20° C.). The layers were allowed to settle for at least 15 min (e.g., 30 min.) while maintaining a temperature of 20° C. to 25° C. The aqueous phase was recharged to the vessel.

A further charge of 5 v of MTBE was added to the vessel while maintaining the temperature below 30° C. The reaction mixture was cooled to about 15° C. to about 25° C. and stirred for about 15 minutes while maintaining the temperature at about 15° C. to about 25° C. (e.g., about 20° C.). The layers were allowed to settle for at least 15 min (e.g., 30 min.) while maintaining a temperature of 20° C. to 25° C.

The separated aqueous phase was recharged to the vessel. 10 v of 2-methyltetrahydrofuran (2-MeTHF) was then charged to the vessel. In an auxiliary vessel was placed a 5M NaOH solution dissolving 0.6p of NaOH and 2.9 v of water. The pH of the aqueous phase in the vessel was adjusted to pH ≥13.0 using the previously prepared 5M NaOH solution, keeping the temperature ≤35° C.

Using maximum agitation, the mixture was stirred for at least 10 minutes at a temperature of about 15° C. to about 25° C. (e.g., 20° C.). The stirring was stopped and the phases were allowed to separate at a temperature of about 20° C. to 25° C.

The organic phase was retained in the vessel. In an auxiliary vessel was charged a 20% w/w NaCl solution dissolving 1.25p of NaCl in 5 v of water. The main vessel containing the organic layer was charged with 5.0 v of the 20% w/w NaCl solution and stirred for least 10 minutes at a temperature of about 15° C. to about 25° C. (e.g., 20° C.). The stirring was stopped and the phases were allowed to separate. The organic phase was retained in the vessel and washed at least three times with 5 v of water. If needed, the organic layer comprising 2-Me THF and Compound (A) was stored at 2-8° C.

Using peroxide strips it was verified that the peroxide content of the IPA (purchased from Ravago Chemicals) to be used was <10 ppm. The organic layer was transferred to a jacketed reactor and heated to about 30° C. to 45° C. (e.g., 35° C.), then concentrated under vacuum to 4-5 residual volumes. 9 v of IPA was charged over the concentrated solution at about 30° C. to 45° C. (e.g., 35° C.), then the mixture was concentrated under vacuum to 4-5 residual volumes. 9 v of IPA charged over the concentrated solution at about 30° C. to 45° C. (e.g., 35° C.), then the mixture was concentrated under vacuum to 4-5 residual volumes. The IPA additions and concentrations were repeated till GC analysis showed 2-MeTHF≤1% w/w to IPA, i.e., solvent exchange from 2-Me THF to IPA was achieved.

In an auxiliary vessel, dissolved 0.28p of benzoic acid in 12 v of IPA at about 20±5° C. The benzoic acid charging amount was calculated based on a w/w assay of Compound (A) (free base). The benzoic acid solution was charged to the main reactor, added further 6 v of IPA to the main reactor. The mixture was heated to a temperature of about 70° C. to about 80° C. (e.g., 76° C.) in about 30 minutes, and heating was continued for an additional 15-30 minutes. The mixture was cooled to about 50° C. to about 55° C. (e.g., about 52° C.) over about 30 to about 60 minutes. 0.005p of seed crystals in 0.05 v IPA was added at about 50° C. to about 55° C. and the mixture was stirred for about 10 to about 15 minutes. The seed crystals were prepared according to procedures described in PCT publication WO 2021/095835. The mixture was then cooled to about 20° C. to about 25° C. over about 200 to about 250 minutes, then stirred at about 20° C. to about 25° C. for about 20 to about 40 minutes. The mixture was then heated to about 62° C. to about 66° C. over about 280 to about 320 minutes, then cooled to about 20° C. to about 25° C. over about 280 to about 320 minutes, and the heat cool cycle was repeated. The mixture was then further stirred for about 6 hours at a temperature of about 20° C. to about 25° C.

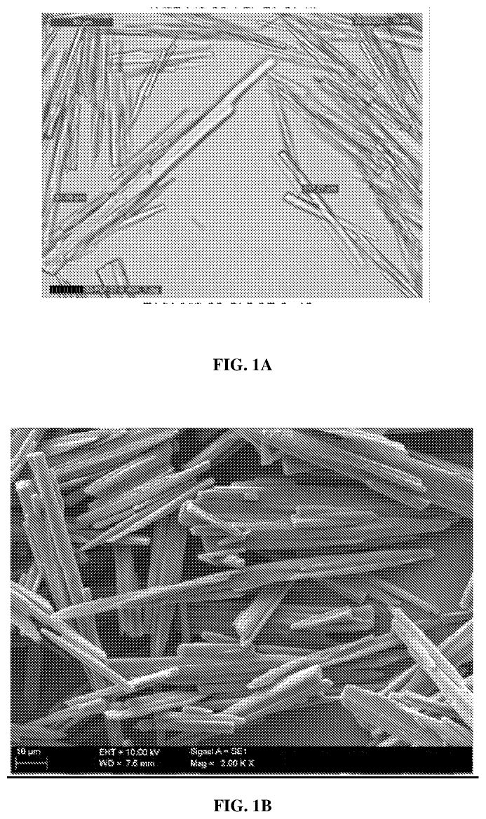

The annealing (i.e., heat cooling cycles) allowed for increased overall yield of the process. The annealing eliminated crusting issues during scale up. Using this process, benzoate salt of Compound (A) with acceptable particle size and morphology was obtained as shown in FIG. 1A and FIG. 1B.

The slurry was filtered and the filter cake was dried in vacuum at up to 40° C. for about 16 h. The sample was analyzed by HPLC to determine if the following impurity is no more than 0.5% a/a.

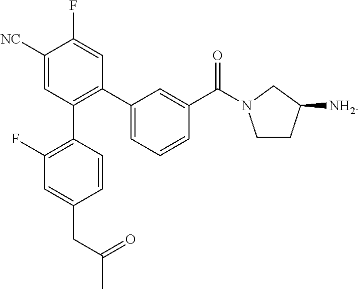 [SMILES: CC(=O)Cc1ccc(-c2cc(C#N)c(F)cc2-c2cccc(C(=O)N3CC[C@H](N)C3)c2)c(F)c1]

The sample was also analyzed to determine if a further impurity at RRT 1.08 was no more than 0.3% a/a, and an additional impurity at RRT 1.15 having the following structure was no more than 0.7% a/a.

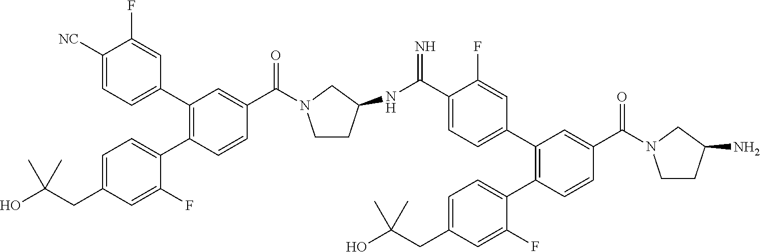 [SMILES: CC(C)(O)Cc1ccc(-c2ccc(C(=O)N3CC[C@H](N)C3)cc2-c2ccc(C(=N)N[C@H]3CCN(C(=O)c4ccc(-c5ccc(CC(C)(C)O)cc5F)c(-c5ccc(C#N)c(F)c5)c4)C3)c(F)c2)c(F)c1]

The HPLC purity method was also used to determine that no more than 0.3% a/a was present for each of the following impurities:

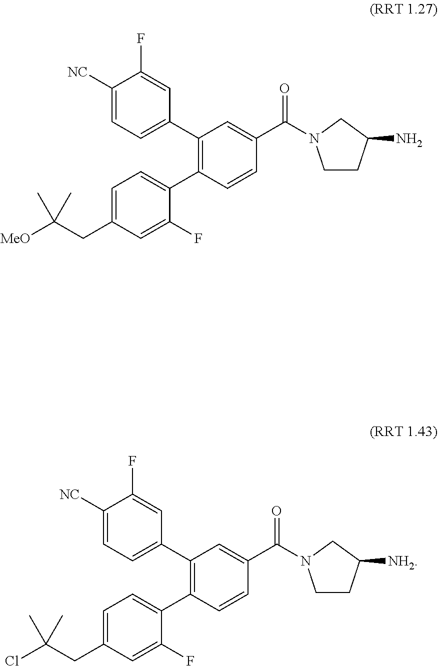 [SMILES: COC(C)(C)Cc1ccc(-c2ccc(C(=O)N3CC[C@H](N)C3)cc2-c2ccc(C#N)c(F)c2)c(F)c1 ; CC(C)(Cl)Cc1ccc(-c2ccc(C(=O)N3CC[C@H](N)C3)cc2-c2ccc(C#N)c(F)c2)c(F)c1]

The HPLC purity method was used to determine that no more than 0.3% a/a was present for any other impurity, and that total impurities were no more than 4.0% a/a. As used herein, a/a refers to the peak area by HPLC.

Optionally, the filter cake is subjected to a second recrystallization from a suitable solvent (e.g., IPA) to obtain the final product.

As described in WO 2021/095835, benzoate salt of Compound (A) has superior solid stability and maintains its purity up to four weeks under the tested conditions.

### Example 2: Preparation of Compound (B) from Compound (C)

Dimethylacetamide (6.5 volumes) was loaded to an appropriately sized reaction vessel, followed by Compound (C). The temperature was adjusted to 0° C. to 5° C. followed by the addition of 0.6 parts Compound (D) and 0.53 parts triethylamine. 1.3 parts of HATU was charged portion-wise and the reaction maintained at about 5° C. until the reaction was determined to be complete by HPLC analysis. Once reaction was complete, isopropyl acetate (10 volumes) was loaded into the reaction mixture followed by water while maintaining the reaction temperature below about 25° C. The aqueous phase was separated and extracted with isopropyl acetate (8 volumes). The combined organic layers were washed sequentially with water (10 volumes), 1N HCl (10 volumes), water (10 volumes), 10% sodium chloride solution (5 volumes) and finally water (10 volumes). The isopropyl acetate solution was recirculated through an AKS-7 charcoal pad until the color was removed. The solvent was distilled to a residual volume of about 5 relative volumes and then MTBE (35 volumes) was added. The temperature of the mixture was adjusted to about 45° C. and then cooled to about 32° C. under high stirring speed. A solid was observed at this point. If a solid is not observed, then crystallization can be initiated by further cooling or the charging of a Compound (B) seed slurry. The seeds of Compound (B) can be prepared using the procedure described in U.S. Pat. No. 10,723,742. Once solids were observed, high speed stirring was maintained for about 4 to 5 hours. The suspension was then cooled to about 10° C. and filtered. The filter cake was washed with MTBE (2×5 volumes) and then dried at about 40° C. to afford Compound (B).

### Biological Examples

### Example A

As described in WO 2021/095835, in Pharmacokinetic (PK) studies in rats, AUC, Cmax(μM) and Tmax(hr) of each of the three drugs, i.e., free form of Compound (A), benzoate salt of Compound (A) and the sorbic acid salt of Compound (A), were calculated. Each drug was administered to the animal at a dose of 32 mg/5 mL/kg. AUC of the free form of Compound (A), the benzoic acid salt of Compound (A) and the sorbic acid salt of Compound (A) were 16.04 μM hr, 16.13 μM hr and 11.64 μM hr. Cₘₐₓ of the three tested compounds were 2.56 μM, 2.68 μM and 2.03 μM. Tₘₐₓ of the three tested compounds were 4.0 hr, 2.7 hr, and 2.3. Thus superior drug adsorption/exposure was achieved with benzoate salt of Compound (A).

### Example B

A Phase 1 Study of safety, pharmacokinetics, and preliminary activity of benzoate salt of Compound (A), as a single agent and in combination with all-trans retinoic acid (ATRA) in subjects with relapsed or refractory (r/r) acute myeloid leukemia (AML) is conducted.

Benzoate salt of Compound (A) is administered as a single agent once daily (QD) on specific days during each 28-day cycle in Part 1. Benzoate salt of Compound (A) is administered QD on specific days during each 28-day cycle in combination with ATRA twice daily (BID) in Part 2. The mode of administration is oral capsules.

Inclusion criteria include and are not limited to a projected life expectancy of at least 12 weeks and in stable condition to complete 1 full cycle (4 weeks) of treatment; histological confirmation of AML by World Health Organization (WHO) 2016 criteria and failure with all other available conventional therapies; peripheral blood or bone marrow blast count >5% at the time of enrollment; disease is refractory to standard induction chemotherapy, including but not limited to anthracycline and cytarabine combination therapy, or has relapsed after anthracycline and cytarabine therapy or stem cell transplant (SCT), or is refractory to or has relapsed after a front-line regimen containing a hypomethylating agent, alone or in combination.

Outcome measures include and are not limited to treatment-emergent adverse events; response rate for complete remission (CR), complete remission with incomplete blood count recovery (CRi), partial remission (PR) and complete remission with partial hematological recovery (CRh); overall survival and other suitable measures.

All patents and other references cited in the specification are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

1. A process for preparing benzoate salt of Compound (A): 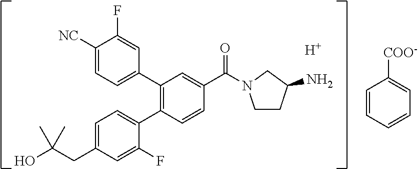
comprising:
(i) extracting Compound (A): 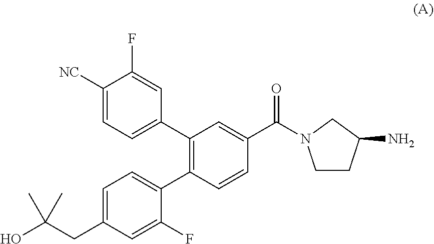 [SMILES: CC(C)(O)Cc1ccc(-c2ccc(C(=O)N3CC[C@H](N)C3)cc2-c2ccc(C#N)c(F)c2)c(F)c1]
into 2-methyl tetrahydrofuran (2-Me THF) from a reaction mixture comprising Compound (A) to obtain a solution of Compound (A) in 2-Me THF;
(ii) conducting a solvent exchange from 2-Me THF to isopropyl alcohol (IPA) to obtain a solution of Compound (A) in IPA; and
(iii) adding benzoic acid to the solution of Compound (A) in IPA under conditions sufficient to obtain the benzoate salt of Compound (A).

2. The process of claim 1, further comprising recrystallizing the benzoate salt of Compound (A).

3. The process of claim 1, where in the peroxide content of the IPA is no more than 10 ppm.

4. The process of claim 1, wherein the Compound (A) in a reaction mixture is obtained by contacting Compound (B): 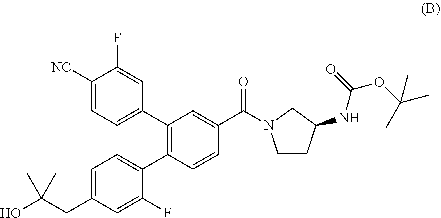 [SMILES: CC(C)(O)Cc1ccc(-c2ccc(C(=O)N3CC[C@H](NC(=O)OC(C)(C)C)C3)cc2-c2ccc(C#N)c(F)c2)c(F)c1]
with aqueous hydrochloric acid thereby providing Compound (A) in the reaction mixture.

5. The process of claim 4, wherein the reaction mixture is heated at a temperature of from about 15° C. to about 50° C.

6. The process of claim 4, wherein the reaction mixture is heated at a temperature of from about 40° C. to about 50° C.

7. The process of claim 4, wherein the reaction mixture is stirred for a period of about 1 hour to about 8 hours.

8. The process of claim 4, further comprising
(i-a) cooling the reaction mixture and adding water and methyl tert butyl ether (MTBE) to the reaction mixture;
(i-b) separating the organic layer comprising MTBE and the aqueous layer;
(i-c) adding 2-Me THF to the aqueous layer from step (i-b) and adding aqueous NaOH to the mixture comprising 2-Me THF and the aqueous layer;
(i-d) separating the organic layer comprising 2-Me THF;
(i-e) washing the organic layer of step (i-d) with 20% w/w NaCl solution;
(i-f) washing the organic layer of step (i-e) with water; and
(i-g) optionally repeating step (i-f) once, or more than once, to obtain a solution of Compound (A) in 2 Me-THF.

9. The process of claim 8, wherein, in step (i-a), the reaction mixture is cooled to a temperature of about 20° C. to about 25° C. and is optionally stirred for a period of about 2 hours to about 24 hours prior to adding water and MTBE.

10. The process of claim 8, wherein, in step (i-c), the aqueous NaOH is added to the mixture to a pH of at least pH 10.

11. The process of claim 8, wherein, after step (i-g), the solution of Compound (A) in 2-Me THF is substantially free of aqueous NaOH.

12. The process of claim 1, wherein step (iii) further comprises heating the reaction mixture to a temperature of about 70° C. to about 80° C. and maintaining the reaction mixture at about 70° C. to about 80° C. for a period of about 30 minutes to about 60 minutes.

13. The process of claim 12, wherein the reaction mixture is cooled to a temperature of about 50° C. to about 55° C.

14. The process of claim 13, wherein a slurry of seed crystal of benzoate salt of Compound (A) in IPA is added to the reaction mixture.

15. The process of claim 14, wherein the reaction mixture is subjected to cooling and heating cycles, then cooled to a temperature of about 16° C. to about 24° C. and stirred at the temperature of about 16° C. to about 24° C. for about 6 hours.

16. The process of claim 15, wherein
(iii-a) the reaction mixture is cooled to a temperature of about 16° C. to about 24° C. over a period of about 200 minutes to about 250 minutes and then stirred at the temperature of about 16° C. to about 24° C. for about 30 minutes;
(iii-b) the reaction mixture is heated to a temperature of about 62° C. to about 66° C. over a period of about 280 minutes to about 320 minutes;
(iii-c) the cooling and heating of steps (iii-a) and (iii-b) is repeated one or two times; and
(iii-d) the reaction mixture is cooled to a temperature of about 16° C. to about 24° C. over a period of about 280 minutes to 320 minutes and stirred at the temperature of about 16° C. to about 24° C. for about 6 hours.

17. The process of claim 1, wherein, the benzoate salt of Compound (A) is recrystallized from ethanol, butanol, IPA, acetonitrile, TBME, ethyl acetate, isopropyl acetate, methylethyl ketone (MEK), proprionitrile, or toluene.

18. The process of claim 1, wherein the benzoate salt of Compound (A) has no more than 0.7% a/a total, of any of the following compounds or a combination thereof:

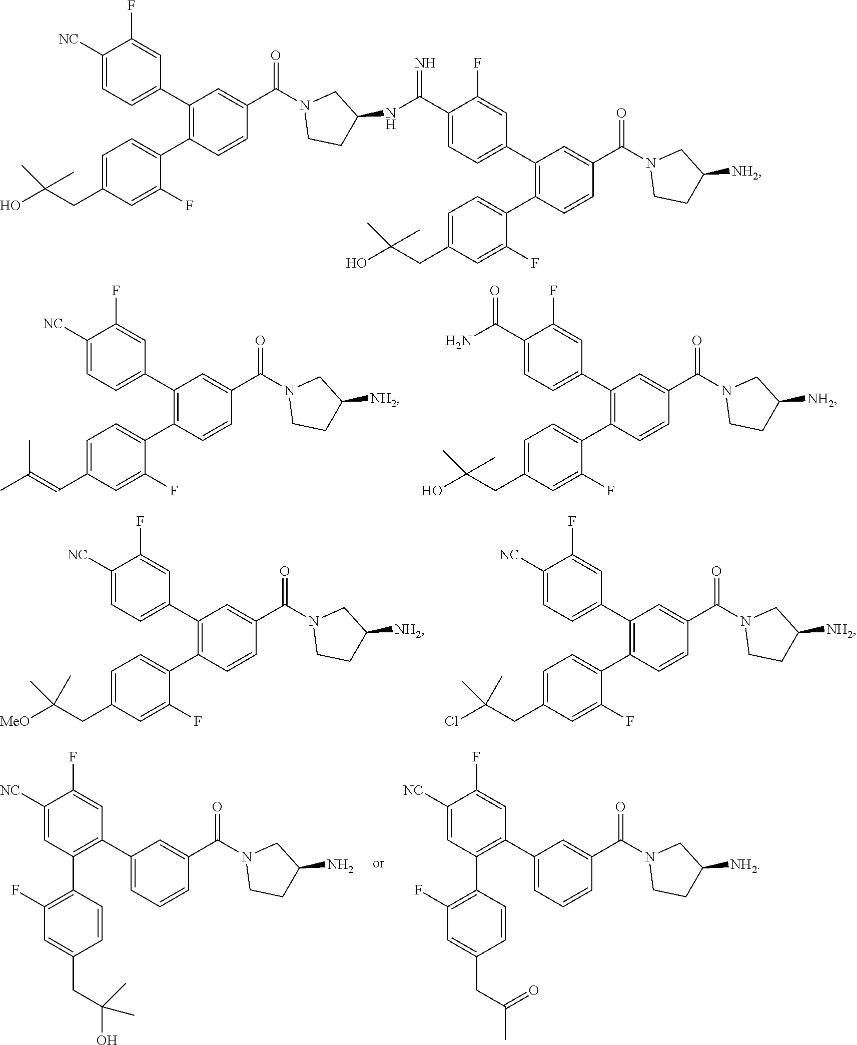

19. A process for preparing benzoate salt of Compound (A): 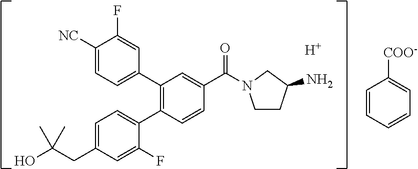
comprising
(i) contacting Compound (B): 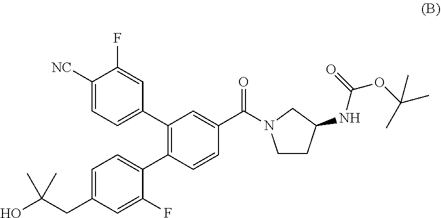 [SMILES: CC(C)(O)Cc1ccc(-c2ccc(C(=O)N3CC[C@H](NC(=O)OC(C)(C)C)C3)cc2-c2ccc(C#N)c(F)c2)c(F)c1]
with aqueous hydrochloric acid to obtain Compound (A): 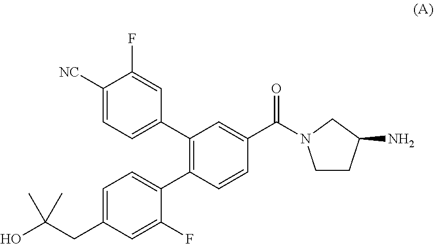 [SMILES: CC(C)(O)Cc1ccc(-c2ccc(C(=O)N3CC[C@H](N)C3)cc2-c2ccc(C#N)c(F)c2)c(F)c1]
(ii) extracting Compound (A) into 2-methyl tetrahydrofuran (2-Me THF) to obtain a solution of Compound (A) in 2-Me THF;
(iii) conducting a solvent exchange from 2-Me THF to isopropyl alcohol (IPA);
(iv) adding benzoic acid to the mixture comprising IPA under conditions sufficient to obtain benzoate salt of Compound (A); and
(v) recrystallizing the benzoate salt of Compound (A).

20. A benzoate salt of Compound (A) prepared according to a process of claim 1.

21. A benzoate salt of Compound (A) comprising no more than 0.7% a/a, total, of any of the following compounds or a combination thereof:

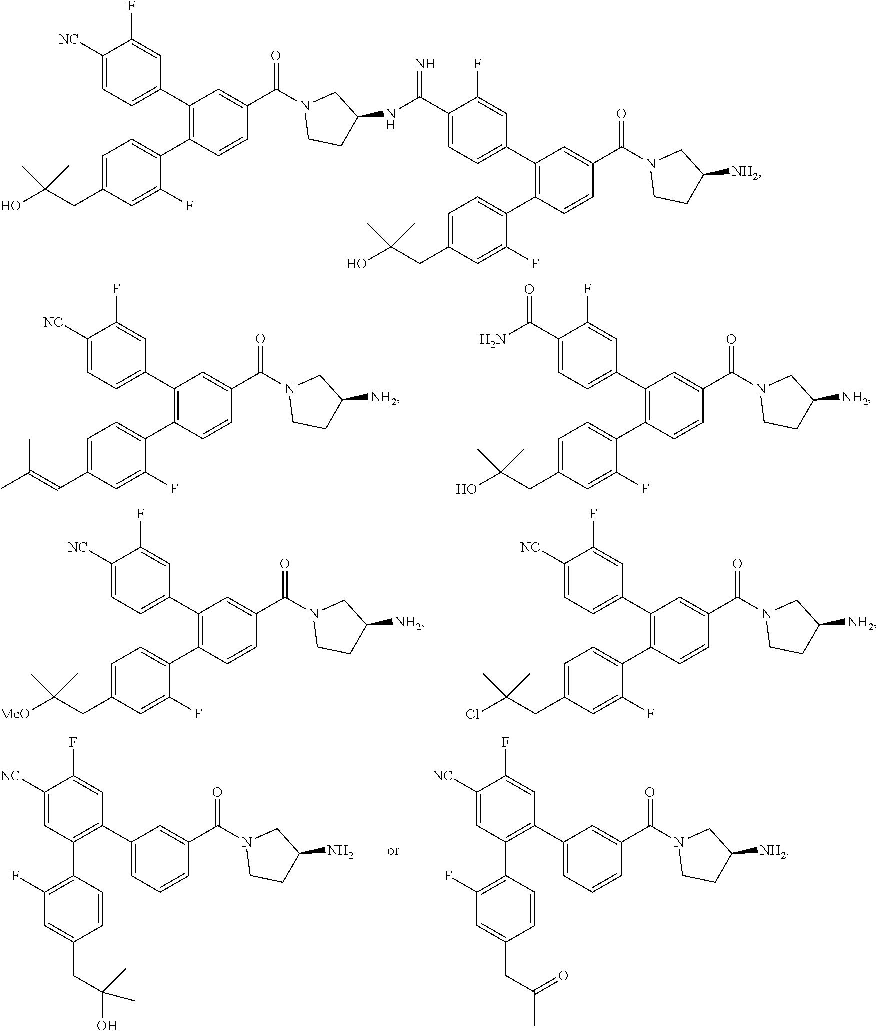

22. A pharmaceutical composition comprising benzoate salt of Compound (A) prepared according to a process of claim 1.

23. A pharmaceutical composition comprising benzoate salt of Compound (A) and comprising no more than 0.7% a/a total, of any of the following compounds or a combination thereof: [chemical structure]
